(19) United States
(12) Patent Application Publication
(10) Pub. No.: US 2022/0088409 A1
(43) Pub. Date: Mar. 24, 2022

(54) SURGICAL SITE DISINFECTION DEVICES

(71) Applicant: Lumitex, Inc., Strongsville, OH (US)

(72) Inventors: Joseph Dombrowski, Strongsville, OH (US); Alan Greszler, Strongsville, OH (US); Michael Kerns, Strongsville, OH (US); Dinusha Thotagamuwa, Strongsville, OH (US)

(21) Appl. No.: 17/480,700

(22) Filed: Sep. 21, 2021

Related U.S. Application Data
(60) Provisional application No. 63/081,399, filed on Sep. 22, 2020.

(51) Int. Cl. A61N 5/06; A61F 13/00; A61B 17/3211; A61B 17/02

(52) U.S. Cl. CPC A61N 5/0624; A61N 5/0603; A61F 13/00063; A61B 17/3211; A61N 2005/0663; A61N 2005/063; A61N 2005/0666; A61N 2005/0661; A61B 17/02

(57) ABSTRACT

A surgical site disinfection device is provided for disinfecting a surgical site. The surgical site disinfection device directs electromagnetic radiation having germicidal properties into the surgical site using a light guide.

### RELATED APPLICATIONS

This application claims the benefit of 63/081,399 filed on Sep. 22, 2020. Which is herein incorporated by reference in its entirety.

### TECHNICAL FIELD

The present disclosure relates generally to disinfection and more particularly to disinfection using light.

### BACKGROUND

Surgical site infection (SSI) represents a significant health risk to patients in both civilian and military hospitals. Combat support hospital surgical suites and triage units are extremely harsh environments that can be filled with various forms of harmful bacteria. Despite best efforts with current practices, each year 1.7 million patients develop a hospital acquired infection and 99,000 patients die.

It is estimated that between 0.5% and 10% of all clean surgeries in the US result in SSI (approximately 275,000 patients/year). Patients who develop SSI are:
60% more likely to spend time in an ICU;
five times more likely to be readmitted;
twice as likely to die;
spend an average of 7 additional days in the hospital; and
roughly double the total healthcare costs.

Approximately 27 million surgical procedures are performed in the United States each year, with up to 5% resulting in SSI. The total annual cost of treating SSIs is projected at $3.2 to $10 billion.

### SUMMARY

Disinfection of surgical operating rooms and equipment typically relies on conventional sterilization techniques, steam, Ethylene Oxide, and some ultraviolet (UV) exposure. There is currently no effective technology for disinfecting the surgical site, on or within the patient. Germicidal UV lamps used to disinfect surgical sites and instruments use a wide range of light wavelengths, causing possible irreparable damage to human cells.

The present disclosure provides a surgical site disinfection device (e.g., a surgical retractor, headlamp, etc.) for disinfecting at the site of the surgery by using wavelengths of light known to inactivate infectious agents.

Both 205-222 nm and 405-408 nm light have been shown to be lethal to most bacteria (such as SARS and MRSA), with little or no damage to the human skin cells. The surgical site disinfection (SSD) device may directly illuminate the surgical site to maximize the disinfectant capability. For example, a range of surgical tools may be adapted to include SSD capabilities.

While several features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a block diagram of an exemplary embodiment of the surgical site disinfection device embodied as a surgical retractor.

FIG. 2 is a three-dimensional (3D) schematic perspective view of the surgical site disinfection device of FIG. 1 positioned adjacent a surgical site.

FIG. 3 is a schematic diagram of an exemplary embodiment of a light guide of the surgical site disinfection device.

FIG. 4 is a perspective view of an exemplary embodiment of the surgical site disinfection device embodied as a surgical retractor.

FIG. 5 is a perspective view of an exemplary embodiment of the light guide and an engaging structure of the surgical site disinfection device.

FIG. 6 is a block diagram of an exemplary embodiment of the surgical site disinfection device including a light source and power source.

FIG. 7 is a schematic diagram of an exemplary embodiment of the surgical site disinfection device embodied as a surgical dressing.

FIG. 8 is a schematic diagram of an exemplary embodiment of the surgical site disinfection device embodied as a scalpel.

FIG. 9 is a block diagram of an exemplary embodiment of a method for disinfecting a surgical site using electromagnetic radiation from a light source having germicidal properties

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

In a general embodiment, the present disclosure provides a surgical site disinfection device for disinfecting a surgical site. The surgical site disinfection device directs electromagnetic radiation (also referred to as light) having germicidal properties into the surgical site using a light guide.

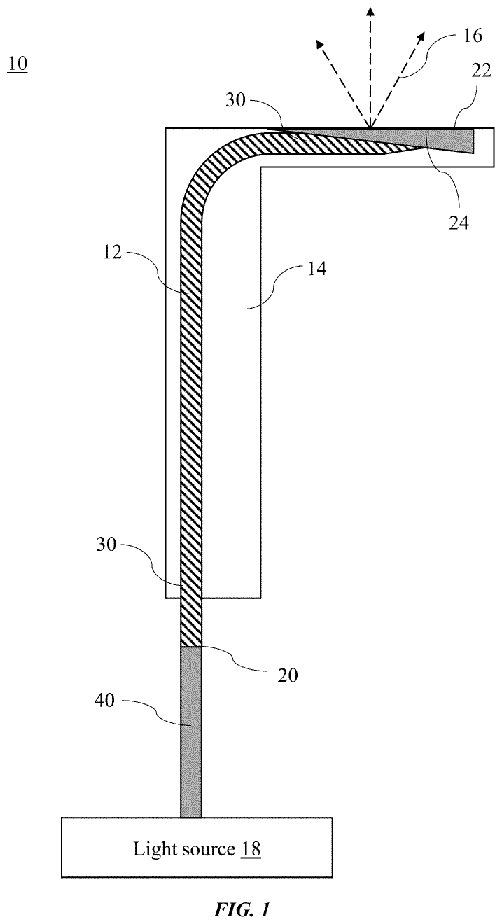

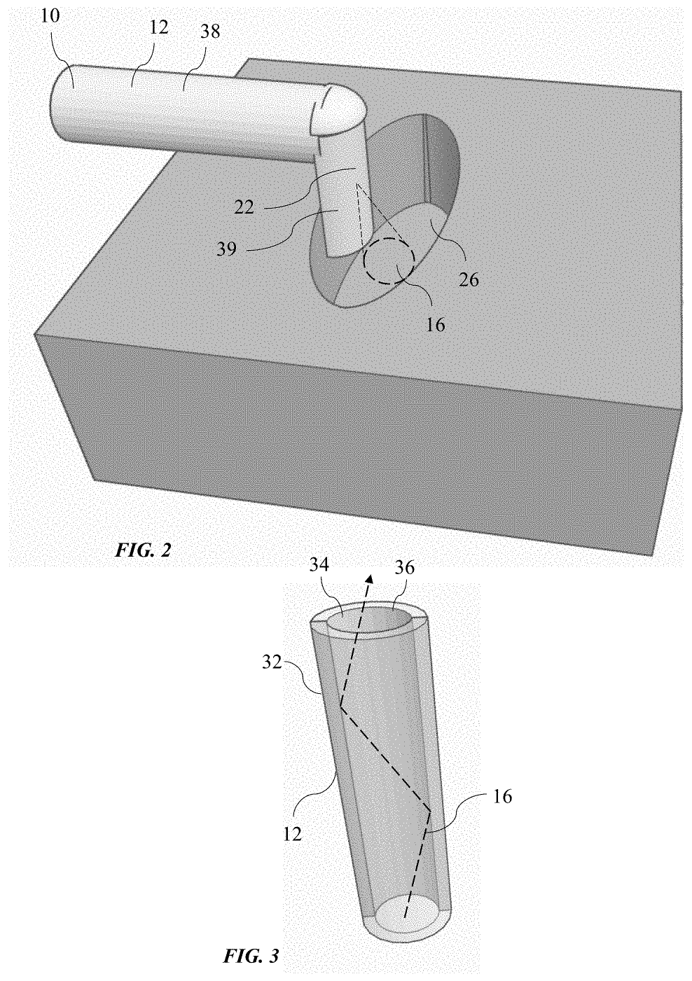

Turning to FIGS. 1 and 2, an embodiment of a surgical site disinfection (SSD) device 10 is shown. The surgical site disinfection device 10 includes a light guide 12 and a housing 14. The light guide 12 receives electromagnetic radiation 16 from a light source 18 at a distal end 20 of the light guide and emits the electromagnetic radiation 16 from a light emitting end 22 of the light guide 12. The housing 14 mechanically supports the light guide, such that electromagnetic radiation 16 emitted from the light emitting end 22 is directed towards a surgical site 24.

After receiving the electromagnetic radiation 16 at the distal end of the light guide 12, the light guide 12 transmits the received electromagnetic radiation 16 from the distal end 12 to the light emitting end 22 of the light guide 12. The transmitted electromagnetic radiation 16 is emitted from the light emitting end 22 of the light guide. The light emitting end 22 may be configured to emit the electromagnetic radiation 16 diffusely, such that the surgical cavity 26 or a portion of the surgical cavity 26 is uniformly illuminated by the electromagnetic radiation 16. The surgical cavity 26 being uniformly illuminated may refer to different illuminated areas of the surgical cavity receiving an optical dose of the electromagnetic radiation 16 that is within 100%, 50%, or 20% of the optical dose received by other areas of the surgical cavity.

In one embodiment, the light emitting end 22 includes light extracting features 24 for extracting light from the light guide 12. The light-extracting features 24 may be used to control the uniformly of illumination provided by the light emitting end 22. The light-extracting features 24 may be any suitable structure for extracting light from the light guide (e.g., to target a specific light output distribution). For example, the light-extracting 24 features may include at least one of surface aberrations, micro-lenses, reflective spots, partial reflective planes, or diffraction gratings. Alternatively or additionally, a diffuser sheet or a 2-D lensing sheet may be (1) placed on an emission surface of the light guide. In one embodiment, the surface aberrations include at least one of a contour of the surface, surface depositions, or surface etchings.

In the embodiment shown in FIG. 3, the light guide includes a tube 32 having a lumen 34 bound by an inner surface 36 that is reflective to the electromagnetic radiation 16, such that the electromagnetic radiation received at the distal end 20 of the light guide 12 is directed towards the light emitting end via reflection by the inner surface 36. For example, the inner surface 36 may be reflective to the electromagnetic radiation 16 due to the inner surface 36 being coated with a reflective material. The reflective material may be any suitable material configured to reflect the electromagnetic radiation. For example, the reflective material may be metallic, such as aluminum.

In one embodiment, the tube 32 may be hollow, such that the lumen 34 is filled with a gas (e.g., air). In another embodiment, the lumen 34 may include a non-gaseous material, such as optical fibers.

The tube 32 may be made from any suitable material. For example, the tube 32 of the light guide 12 may be made from plastic, metal, etc. When the tube 32 is made from a material that is not reflective to the electromagnetic radiation (e.g., less than 50% reflective the electromagnetic radiation 16), then the inner surface 36 may be coated with the reflective to material as described above. For example, in one embodiment, the tube 32 is made from plastic and the inner surface 36 is coated with aluminum (e.g., via vapor deposition).

The housing 14 mechanically supports a supported portion 30 of the light guide 12, such that the electromagnetic radiation 16 emitted from the light emitting end 22 is directed towards the surgical site 26. For example, in the embodiment shown in FIGS. 1 and 2, the housing is a surgical retractor and the light guide 12 is contained within the housing 14. However, in alternative embodiments the light guide 12 may be more external to the housing 14, such that the supported portion 30 of the light guide 12 is a smaller percentage of the light guide 12. For example, the supported portion 30 of the light guide 12 may be only the portion of the light guide 12 that is located close to the light emitting end 22.

In one embodiment, the housing 14 is at least partially made of an optical material capable of transmitting the electromagnetic radiation 16. For example, at least a portion of the housing 14 may act as a light guide for directing light from the light source 18 to the surgical site 26.

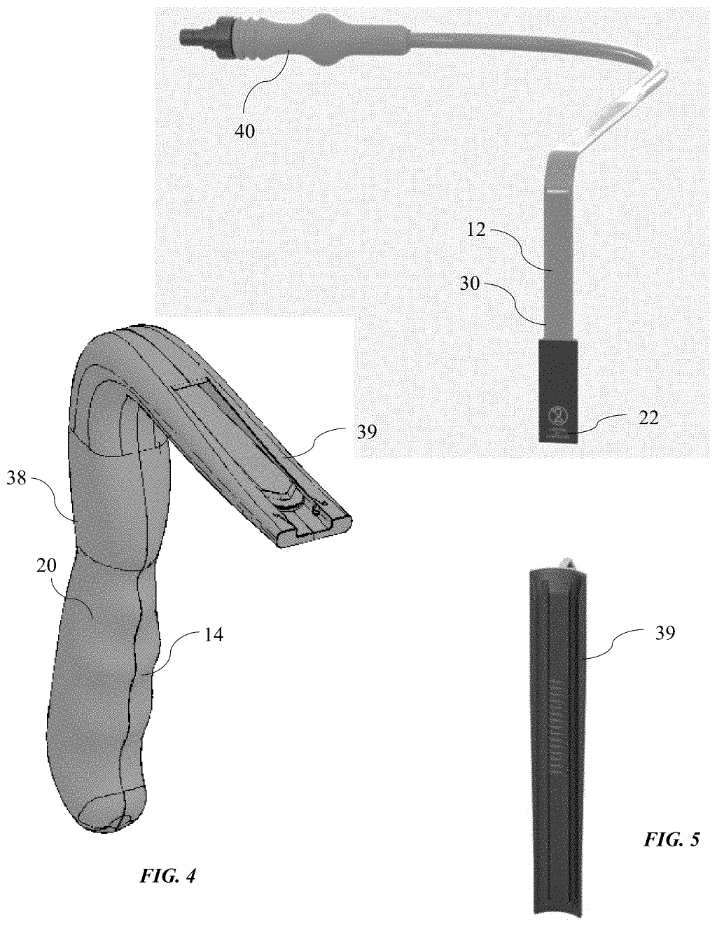

In the embodiment shown in FIG. 4, the housing 14 includes a supporting structure 38 and an engaging structure 39 mechanically supported by the supporting structure 38. The engaging structure 39 interacts with the surgical site 24. For example, in embodiments including a surgical retractor, the engaging structure includes the portion of the surgical retractor that pull/pushes to keep the surgical site open and the handle includes the supporting structure 38. FIG. 4 shows the housing 14 without the light guide 12.

In the embodiment shown in FIG. 5, the light guide 12 is shown separate from the supporting structure 39 before the light guide 12 is inserted into the supporting structure 39. At least one of the engaging structure 39 or the supporting structure 38 mechanically supports a supported portion 30 of the light guide 12, such that the electromagnetic radiation 16 emitted from the light emitting end 22 is directed towards the surgical site 24. For example, the supporting structure 39 may include a channel for receiving the light guide 12 as shown in FIGS. 4 and 5.

As shown in FIG. 1, the surgical site disinfection device 10 may include an intermediate optical guide 40 that is optically connected to the light source 18 and the light guide 12, such that electromagnetic radiation 16 emitted by the light source 18 is received by the intermediate optical guide 40 and transmitted to the distal end 20 of the light guide 12. The intermediate optical guide 40 may be formed from any suitable structure capable of acting as a light guide. For example, the intermediate optical guide 40 may include glass fibers.

The electromagnetic radiation 16 may include any suitable wavelengths of light. For example, the electromagnetic radiation may include at least one of 222 nm or 405 nm. As an example, the electromagnetic radiation may include at least one of 405+/−25 nm or 222+/−5 nm.

In addition to disinfecting light, the electromagnetic radiation may additionally include photobiomodulation light. For example, the photobiomodulation wavelengths may be wavelengths (e.g., 600-1200 nm) configured to stimulate wound healing. As an example, the light source 18 may include multiple light emitters. One or more of the light emitters may emit the disinfecting light while other light emitter(s) emit the photobiomodulation light.

The light source 18 may be any suitable structure for emitting electromagnetic radiation. For example, the light source 18 may include one or more light emitting diodes (LEDs), organic LEDs (OLEDs), micro-LEDs, laser diodes, mini-LED, quantum dot (QD)-conversion, phosphor conversion, excimer lamps, multi-photon combination, or SLM wavefront manipulation.

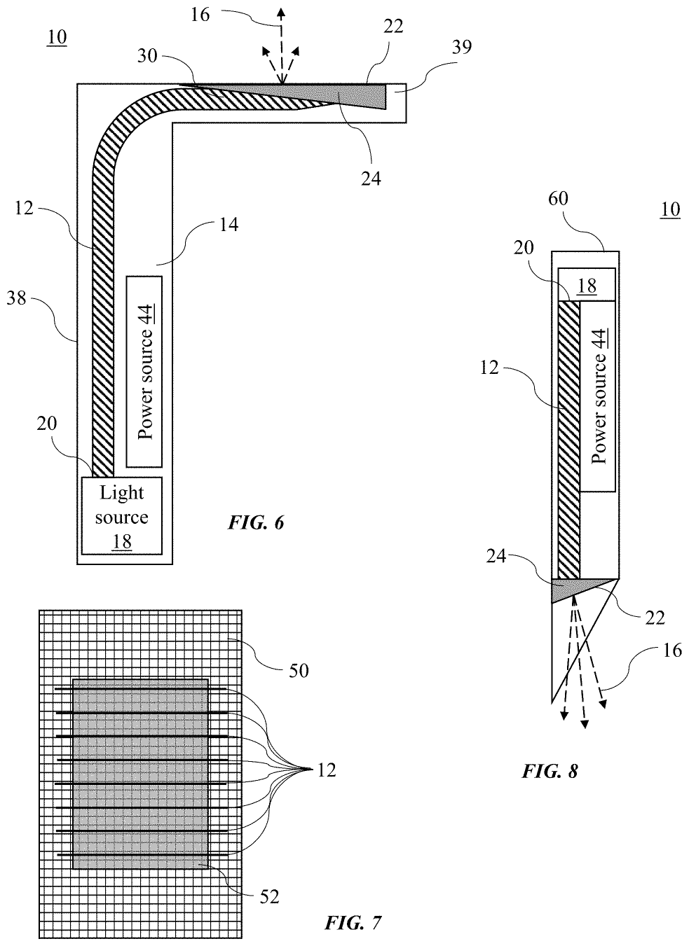

In the embodiment shown in FIG. 6, the surgical site disinfection 10 includes the light source 18. For example, the light source 18 may be mechanically supported by the housing 14. That is, the surgical site disinfection device 10 may include a housing 14 that physically supports the light source 18. Alternatively, the housing 14 may be remotely located from the light source 18 and the housing 14 may instead physically support the light guide. Even when the housing physically supports the light source, the surgical site disinfection device 10 may additionally include a light guide 12 that receives the electromagnetic radiation 16 (also referred to as disinfecting light) from the light source 18 and that is shaped and/or positioned to emit the disinfecting light onto the surgical site.

The surgical site disinfection (SSD) 10 may also include a power source 44. The power source is configured to store and transmit electric power and is electrically connected to the light source 18. The power supply 44 may be any suitable power storage device. For example, the power supply 44 may be a rechargeable battery.

In the embodiment shown in FIG. 7, the housing 14 includes a surgical dressing 50. The surgical dressing 50 may include a photocatalytic material 52 configured to emit reactive oxygen species when illuminated by the electromagnetic radiation 18. The light guide 12 may be positioned relative to the surgical dressing 50 such that the electromagnetic radiation 16 emitted from the light emitting end 22 illuminates the photocatalytic material 52, causing the photocatalytic material to emit the reactive oxygen species. For example, the surgical dressing 50 may include a flexible, large area light source. For example, the light source 18 may be formed from an array of light emitters (e.g., a light emitting diode (LED), micro-LED, organic LED (OLED), a fiber optic webbing, or a flexible flat fiber patch). The surgical dressing could be used during surgery (e.g., around the wound incision site) and after surgery.

In one embodiment, the surgical dressing 50 includes a photocatalytic material 52. When the photocatalytic material 52 is illuminated by the disinfecting light, the photocatalytic material 52 creates reactive oxygen species to disinfect tissues adjacent to the surgical site disinfection device 10. In this embodiment, the light source 18 may emit UV light (e.g., UVA, UVC, and/or light having a wavelength below 385) as excitation light to cause the photocatalytic material 52 to create the reactive oxygen species.

In another embodiment, the surgical site disinfection device 10 is a wearable lighted personal protective equipment (PPE). For example, the PPE could be a glove, gown, cap, mask, etc. made of photocatalytic materials or lined with light sources that directly emit disinfecting light. For example, the light sources could be woven into the PPE or used to illuminate a sheet light guide material. For example, the PPE may be a glove (e.g., latex glove) that is placed over top of the light source(s). The glove may be non-permeable to fluids and germs, but at least partially transparent to the disinfecting light.

In the embodiment shown in FIG. 8, the housing 14 includes a scalpel 60 and the light emitting end 22 is positioned to direct the electromagnetic radiation 16 emitted from the light emitting end 22 onto tissue 62 cut by the scalpel 60. The housing 14 is not limited to a scalpel but may include any surgical implement (e.g., a cutting tool) used during surgery. For example, the surgical implement any cutting tool that focus the electromagnetic radiation at the site where tissue is being cut. In this way, the tissue may be disinfected prior to and during cutting.

In still another embodiment, the surgical site disinfection device 10 may be embodied as a suture made of optical fiber capable of transmitting the electromagnetic radiation 16 from the light source 18 and emitting the electromagnetic radiation 16 along a length of the suture. For example, the suture may be made of glass and the light source and power supply (e.g., battery) may be located in a bandage configured to overlay and interface optically with the suture.

In another embodiment, the surgical site disinfection device 10 may be integrated into a standard operating room (OR) overhead light. For example, the surgical site disinfection device 10 may be received by a standard OR overhead light in the same manner as a standard light bulb.

In a further embodiment, the surgical site disinfection device 10 may be a bendable light that is configured to fix to an operating table or similar surface. For example, the surgical site disinfection device 10 may be bendable and retain position to provide disinfecting light to a surgical site.

The surgical site disinfection device 10 may also include a light blocking layer to limit exposure of the disinfecting light to the desired treatment area. For example, the surgical site disinfection device 10 may include an aperture to limit a spread of light emitted by the surgical site disinfection device 10. Alternatively or additionally, the surgical site disinfection device 10 may include a sheet like material for covering a portion of a patient. The sheet like material may be perforated or cuttable, such that a through hole is created in the sheet like material. The through hole may then be positioned such that only the desired treatment area is exposed.

The surgical site disinfection device 10 may be utilized in conjunction with a machine vision system. For example, the machine vision system may be communicatively coupled to the surgical site disinfection device 10 to control the area illuminated by the light source. The machine system may include a camera to image an area illuminated by the light source. The machine vision system may also be configured to receive an input from a user indicating a desired area to be illuminated. For example, a user may place visible fiducials to designate the desired area. The machine vision system may then control the surgical site disinfection device 10 such that only the desired area is illuminated.

A gel material may also be used with the surgical site disinfection device 10. For example, the gel material may be index of refraction matching to improve light coupling (e.g., between the light source and the light guide, the light guide and tissue, and/or the light source and the tissue). As another example, the gel material may include light block particulates for masking sensitive areas. In still another example, the gel material may contain at least one of wavelength converting nanoparticles for converting light to a particular wavelength or photocatalytic particles.

As described above, the surgical site disinfection device may include circuitry configured to control the light source. The circuitry may be configured to control the light source to provide a programmable, dynamic light intensity to provide optimal disinfection exposure per a prescribed schedule. For example, the prescribed schedule may be stored in memory (e.g., non-transitory computer readable medium) communicatively coupled to the circuitry. As an example, the light source may include an array of light emitters and/or optics for controlling a spot size/shape of the disinfecting light. The circuitry may be configured to control the light emitters and/or the optics per the prescribed schedule. In one embodiment, the surgical site disinfection device 10 comprises a cannula configured to receive and disinfect surgical tools. For example, the cannula may be constructed of light transmitting materials or lined with light emitters of the light source.

As another example, the surgical site disinfection device 10 may be an endoscope (or catheter). The endoscope may be configured to emit the disinfecting light from a tip of the endoscope. Alternatively or additionally, the endoscope may be configured to emit the disinfecting light from a surface of the endoscope that comes in contact with patient tissues (e.g., the external surface of the endoscope). For example, the surgical site disinfection device 10 may be a light transmitting tubing able to disinfect its own surfaces as well tissue it contacts.

In another embodiment, the surgical site disinfection device 10 may be a grommet for sealing and disinfecting internally insertable tubing. The grommet may be constructed of light transmitting materials or lined with light emitters.

The surgical site disinfection device 10 may include a communication interface for communicating with other medical devices. For example, the communication interface may utilize a communication protocol for coordinating optimal disinfection dose with other medical devices.

In one embodiment, the surgical site disinfection device 10 may be embodied as a headlamp acting as a head worn source of disinfecting light. For example, as a surgeon wearing the headlamp focuses her attention on a surgical site, the head worn lamp provides disinfecting light that illuminates the surgical site. As is described below, the head lamp may apply the disinfecting light directly or photocatalytically.

In one embodiment, the surgical site disinfection device 12 includes circuitry and sensor(s). The circuitry is communicatively coupled to the sensor(s) and is configured to control operation of the light source based on an input from the sensor(s). For example, the sensor(s) may be a motion sensor (e.g., a gyroscope and accelerometer). The circuitry may determine when the surgeon has stopped moving the surgical site disinfection device to focus on a location. As an example, when the movement detected by the sensor(s) is below a movement threshold for a time threshold (i.e., a duration of time), then the circuitry may cause the light source to emit light. Conversely, when the sensor(s) has detected movement greater than the movement threshold within a time duration less than the time threshold, then the circuitry may prevent the light source from emitting light. In this way, illumination by the disinfecting light of undesirable surfaces (e.g., other medical personnel) may be minimized.

In one embodiment, the surgical site disinfection device 10 may be sterilized and disposable with the light source 18 built it. For example, to work in combat areas, the surgical site disinfection device 10 may be single use, sterilized, and battery operated to allow for reduced surgery prep time.

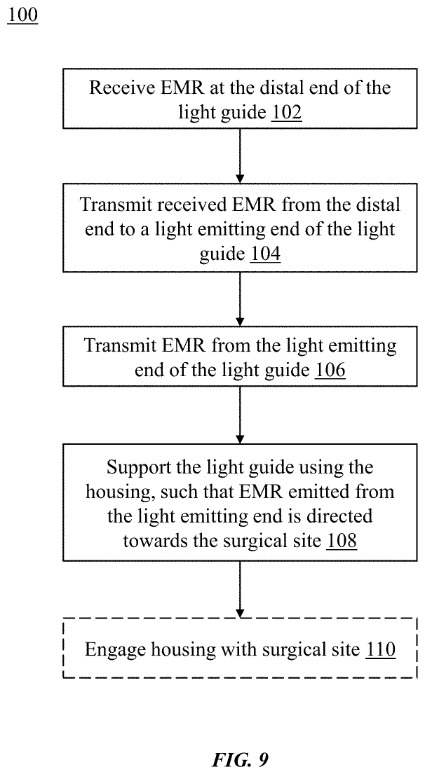

In FIG. 9, an exemplary embodiment of a method 100 for disinfecting a surgical site 26 using electromagnetic radiation 16 from a light source 18 having germicidal properties is shown. In step 102, the electromagnetic radiation 16 emitted by the light source 18 is received at the distal end 20 of the light guide 12. In step 104, the received electromagnetic radiation 16 is transmitted from the distal end 20 to a light emitting end 22 of the light guide 12. In step 106, the transmitted electromagnetic radiation 16 is emitted from the light emitting end 22 of the light guide 12. In step 108, a supported portion of the light guide is mechanically supported using the housing 14, such that the electromagnetic radiation 16 emitted from the light emitting end 22 is directed towards the surgical site 26. In optional step 110, the housing 14 mechanically engages with the surgical site 26.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A surgical site disinfection device for disinfecting a surgical site using electromagnetic radiation from a light source having germicidal properties, the surgical site disinfection device comprising:
a light guide configured to:
receive the electromagnetic radiation emitted by the light source at a distal end of the light guide;
transmit the received electromagnetic radiation from the distal end to a light emitting end of the light guide; and
emit the transmitted electromagnetic radiation from the light emitting end of the light guide;
a housing configured to mechanically support a supported portion of the light guide, such that the electromagnetic radiation emitted from the light emitting end is directed towards the surgical site.

2. The surgical site disinfection device of claim 1, wherein the housing includes a surgical retractor.

3. The surgical site disinfection device of claim 1, wherein the light guide comprises a tube having a lumen bound by an inner surface that is reflective to the electromagnetic radiation, such that the electromagnetic radiation received at the distal end of the light guide is directed towards the light emitting end via reflection by the inner surface.

4. The surgical site disinfection device of claim 3, wherein the inner surface is reflective to the electromagnetic radiation due to the inner surface being coated with a reflective material.

5. The surgical site disinfection device of claim 4, wherein the reflective material includes aluminum.

6. The surgical site disinfection device of claim 3, wherein the tube is hollow.

7. The surgical site disinfection device of claim 3, wherein the tube of the light guide is made from plastic.

8. The surgical site disinfection device of claim 1, further comprising an intermediate optical guide configured to be optically connected to the light source and the light guide, such that electromagnetic radiation emitted by the light source is received by the intermediate optical guide and transmitted to the light guide.

9. The surgical site disinfection device of claim 8, wherein the intermediate optical guide includes glass fibers.

10. The surgical site disinfection device of claim 1, wherein a wavelength of the electromagnetic radiation includes at least one of 222 nm or 405 nm.

11. The surgical site disinfection device of claim 1, further comprising the light source, wherein the light source is mechanically supported by the housing.

12. The surgical site disinfection device of claim 11, further comprising a power source electrically connected to the light source.

13. The surgical site disinfection device of claim 1, wherein the housing includes a surgical dressing.

14. The surgical site disinfection device of claim 13, wherein:
the surgical dressing includes a photocatalytic material configured to emit reactive oxygen species when illuminated by the electromagnetic radiation; and
the light guide is positioned relative to the surgical dressing such that the electromagnetic radiation emitted from the light emitting end illuminates the photocatalytic material, causing the photocatalytic material to emit the reactive oxygen species.

15. The surgical site disinfection device of claim 1, wherein the housing includes a scalpel and the light emitting end is positioned to direct the electromagnetic radiation emitted from the light emitting end onto tissue cut by the scalpel.

16. A method for disinfecting a surgical site using electromagnetic radiation from a light source having germicidal properties, the method comprising:
receiving the electromagnetic radiation emitted by the light source at a distal end of a light guide;
transmitting the received electromagnetic radiation from the distal end to a light emitting end of the light guide;
emitting the transmitted electromagnetic radiation from the light emitting end of the light guide;
mechanically support a supported portion of the light guide using a housing, such that the electromagnetic radiation emitted from the light emitting end is directed towards the surgical site.

17. The method of claim 16, further comprising the housing mechanically engaging with the surgical site, wherein:
the transmitting of the received electromagnetic radiation from the distal end to the light emitting end of the light guide includes reflecting the electromagnetic radiation off of an inner surface of a hollow lumen of the light guide.

18. A surgical retractor for disinfecting a surgical site using electromagnetic radiation from a light source having germicidal properties, the surgical site disinfection device comprising:
a housing including a supporting structure and an engaging structure mechanically supported by the supporting structure, wherein the engaging structure is configured to interact with the surgical site;
a light guide configured to:
receive the electromagnetic radiation emitted by the light source at a distal end of the light guide;
transmit the received electromagnetic radiation from the distal end to a light emitting end of the light guide; and
emit the transmitted electromagnetic radiation from the light emitting end of the light guide;
wherein at least one of the engaging structure or the supporting structure is configured to mechanically support a supported portion of the light guide, such that the electromagnetic radiation emitted from the light emitting end is directed towards the surgical site.

19. The surgical retractor of claim 18, wherein the light guide comprises a tube having a lumen bound by an inner surface that is reflective to the electromagnetic radiation, such that the electromagnetic radiation received at the distal end of the light guide is directed towards the light emitting end via reflection by the inner surface.

20. The surgical retractor of claim 18, further comprising the light source and a power source electrically connected to the light source, wherein the light source is mechanically supported by the housing.
